(19) United States
(12) Patent Application Publication
(10) Pub. No.: US 2025/0352465 A1
(43) Pub. Date: Nov. 20, 2025

(54) MICRONEEDLE DEVICE

(71) Applicant: DARWIN PRECISIONS CORPORATION, Hsinchu County (TW)

(72) Inventors: Jyun-Yi Yu, Hsinchu County (TW); Yun-Pei Yang, Hsinchu County (TW)

(21) Appl. No.: 19/026,596

(22) Filed: Jan. 17, 2025

(30) Foreign Application Priority Data
May 14, 2024 (TW) 113117686

(51) Int. Cl. A61K 9/00; A61K 47/34; A61K 47/36; A61K 47/69

(52) U.S. Cl. CPC A61K 9/0021; A61K 47/34; A61K 47/36; A61K 47/6957

(57) ABSTRACT

A microneedle device including a base and a plurality of microneedles is provided. The microneedles are arranged on a surface of the base, wherein the material of the base and the microneedles include polyglutamic acid and pullulan. The ratio of the weight of polyglutamic acid to the weight of pullulan is ranged from 0.1 to 0.9. The microneedle device dissolves quickly and is easy to prepare.

### FIELD OF THE INVENTION

The present invention relates to a transdermal delivery device, in particular to a microneedle device.

### BACKGROUND OF THE INVENTION

Common drug delivery methods include oral administration, subcutaneous injection, and transdermal delivery, wherein transdermal delivery allows the drug to be absorbed through the skin and into the blood circulatory system, therefore transdermal delivery can make the drug concentration in the blood more stable than oral administration and subcutaneous injection and has benefit to avoid the pain and wound infection during the injection. The effect of controlling the transdermal delivery of drugs can be adjusted by the design of the dosage form, wherein the microneedle device can provide the most direct way because the tens of thousands of microneedles (MN) allow macromolecules and drugs to penetrate the stratum corneum and enter the skin through micro-pores, thus eliminating the obstruction of transdermal delivery caused by the stratum corneum.

The microneedles of the microneedle device described above can be made into solid, coated, hollow, or dissolvable microneedles from various materials such as silicon, stainless steel, carbohydrates, and polymers, each with different properties and applications. The above-mentioned dissolvable microneedles have the property of promoting the rapid release of macromolecules, but they often face the problems of slow dissolution rate and difficulty of manufacturing.

### SUMMARY OF THE INVENTION

The invention provides a microneedle device by adjusting the material and proportional content of the material in the microneedles. The adjusting material can make the microneedle device dissolve quickly and has manufacturing feasibility.

The microneedle device provided by the present invention includes a base and a plurality of microneedles. The plurality of microneedles is arranged on the surface of the base, wherein the material of the base and the plurality of microneedles include polyglutamic acid and pullulan; furthermore, a ratio of a weight of the polyglutamic acid to a weight of the pullulan is ranged from 0.1 to 0.9.

In one embodiment of the present invention, the material of the base and the microneedles further comprises a functional ingredient, the functional ingredient is selected from a group consisting of at least one of hyaluronic acid, salicylic acid, vitamin B5, vitamin C, vitamin E, nicotinic acid, sulfur, Centella asiatica, ceramide, glycerin, GLP-1 receptor agonist, insulin and acetaminophen.

In one embodiment of the present invention, the aforementioned polyglutamic acid accounts for 1% to 50% of the weight of the microneedle device, and the pullulan accounts for 1% to 90% of the weight of the microneedle device.

In one embodiment of the present invention, a molecular weight of the polyglutamic acid is between 400 kDa and 1,000 kDa.

In one embodiment of the present invention, a molecular weight of the pullulan is between 200 kDa and 1,000 kDa.

In one embodiment of the present invention, each of the microneedles has a height in a direction perpendicular to the surface, and the height is between 100 μm and 1,500 μm.

In one embodiment of the present invention, each of the microneedles has a height in a direction perpendicular to the surface, and the height is reduced to less than or equal to half of the height before attachment after the microneedle device is attached to the skin for 60 minutes.

In one embodiment of the present invention, each of the microneedles has a height in a direction perpendicular to the surface, and the height is reduced to less than or equal to one-third of the height before attachment after the microneedle device is attached to the skin for 120 minutes.

The present invention microneedle device contains polyglutamic acid and pullulan, and the ratio of the weight of the polyglutamic acid to the weight of the pullulan is ranged from 0.1 to 0.9. Therefore, the microneedle device has a fast dissolution rate and possesses the manufacturing feasibility.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic diagram of a microneedle device in an embodiment of the present invention;

FIG. 2 is a schematic diagram of the microneedle device attached to the skin according to an embodiment of the present invention; and

FIG. 3 is a diagram showing the experimental results of the microneedle device in an embodiment of the present invention and the soluble microneedle device of the prior art.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

A common dissolvable microneedle device is made by: pouring a liquid containing a polymer material (hereinafter referred to as liquid) into a master mold with grooves corresponding to the plurality of microneedles and then drying the liquid to form the microneedle device. The liquid is composed of solutes and solvents. Because the solvents evaporate almost completely when drying, the microneedle device is essentially composed of solutes. For example, the solute in the liquid is a soluble polymer material and the solvent is generally water, but the present invention does not make specific restrictions on this. The master mold may be made of a material such as silica gel, plastic, or metal, and the present invention is not limited to this.

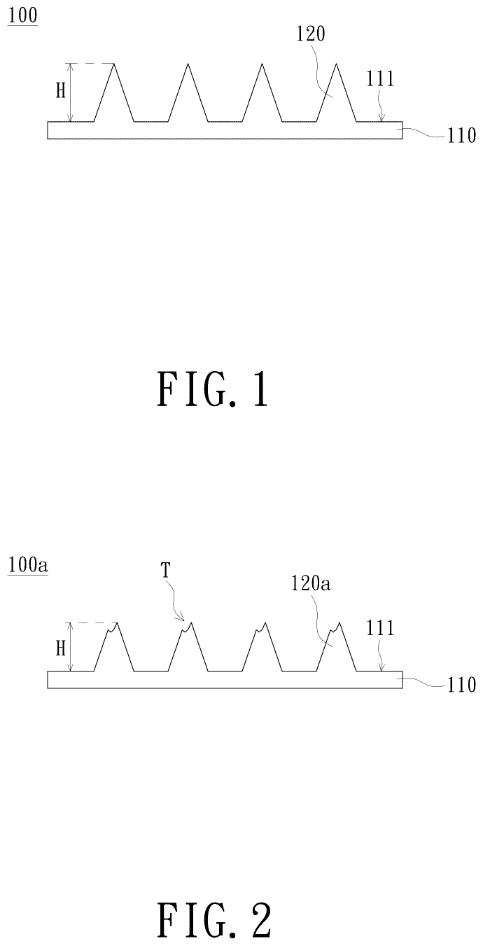

FIG. 1 is a schematic diagram of a microneedle device in an embodiment of the present invention. Please refer to FIG. 1. The microneedle device 100 includes a base 110 and a plurality of microneedles 120. The plurality of microneedles 120 is arranged on the surface 111 of the base 110. The material of the base 110 and the plurality of microneedles 120 includes polyglutamic acid (γ-PGA) and pullulan, and the ratio of the weight of the polyglutamic acid to the weight of the pullulan ranges from 0.1 to 0.9. In the present embodiment, each of the microneedles 120 of the microneedle device 100 has a height H perpendicular to the surface 111, the height H can be, for example, ranged between 100 μm and 1,500 μm, such as, for example, 100 μm, 200 μm, 300 μm, 400 μm, 500 μm, 600 μm, 700 μm, 800 μm, 900 μm, 1,000 μm, 1,100 μm, 1,200 μm, 1,300 μm, 1,400 μm, and 1,500 μm, and the present invention does not make specific restrictions on this. The polyglutamic acid has the advantage of a high dissolution rate and is suitable as the material for the microneedle device 100. In addition, the polyglutamic acid with an appropriate amount of the pullulan can prevent the microneedle device 100 from melting by moisture, and achieve that the microneedle can be dissolved more than 50% in 1 hour and the microneedle can be dissolved more than 70% in 2 hours.

It should be noted that the “microneedle can be dissolved more than 50% in 1 hour” means that the height H of the residual microneedle can be reduced, observed by microscopy, to half or less of the original height after the microneedle device is attached to the skin for 1 hour and then removed from the skin. For example, if the height H of the microneedles before attachment is 200 μm, the height H of the microneedles is reduced to 100 μm or less after 1 hour of attachment to the skin. In the same way, the “microneedle can be dissolved more than 70% after 2 hours” means that the height H of the residual microneedle can be reduced to 30% or less of the original height after the microneedle device is attached to the skin for 2 hours and then removed from the skin. Specifically, the microneedle device 100 shown in FIG. 1 is attached to the skin and begins to dissolve, and the microneedle device 100 becomes the microneedle device 100a shown in FIG. 2 after a period of time. The microneedles 120a of the microneedle device 100a in the present embodiment have, for example, high and low fluctuations at the top T away from the base 110, and the farthest point of the top T along the direction perpendicular to the surface 111 (i.e., the farthest point of the microneedles 120a from the surface 111) is taken as the height H of the microneedles 120a when measuring the height H of the microneedles 120a.

In addition, the viscosity of the liquid can affect the effect of coating the liquid on the master mold. For example, if the viscosity of the liquid is too high, it will affect the structure of the microneedle device, including poor flatness of the base (or membrane surface), incomplete needle structure of the microneedles, and poor integrity of the microneedle device after demolding (e.g., rupturing during demolding). It is to be noted that increasing the content of the pullulan is unlikely to increase the viscosity of the liquid, so the problem in that the viscosity of the liquid is too high therefore making it difficult for the liquid to evenly cover the master mold can be avoided.

In this embodiment, the molecular weight of the polyglutamic acid is between 400 kDa and 1,000 kDa, such as 400 kDa, 500 kDa, 600 kDa, 700 kDa, 800 kDa, 900 kDa, or 1000 kDa. The molecular weight of the pullulan is, for example, between 200 kDa and 1,000 kDa, such as 200 kDa, 300 kDa, 400 kDa, 500 kDa, 600 kDa, 700 kDa, 800 kDa, 900 kDa, or 1,000 kDa. This allows the liquid to have a more appropriate viscosity to further enhance the effect of attaching the liquid to the surface of the master mold. In this embodiment, the larger the molecular weight of the polyglutamic acid, the higher the viscosity of the liquid (that is, the liquid becomes thicker). In this embodiment, the microneedles are cone-shaped or pyramid-shaped, for example, but the present invention is not limited to this.

Table 1 shows the microneedle devices made using a liquid containing polyglutamic acid and pullulan and the evaluation of the flatness of the base of each microneedle device, the integrity of the microneedle device after demolding, the needle structure and the dissolution rate of the microneedles. The “ingredient 1” in Table 1 is the weight percentage of the polyglutamic acid in the liquid. The “ingredient 2” is the weight percentage of the pullulan in the liquid. The “ratio” is the ratio of the weight of the polyglutamic acid to the weight of the pullulan, i.e., the value obtained by dividing the weight of the polyglutamic acid by the weight of the pullulan sugar, rounded to two decimal places. The “dissolving effect after 2 hours” means observing, by using a microscope, whether the microneedles are completely dissolved or there are residues after the microneedle device is attached to the skin for 2 hours and then is removed from the skin, wherein the height H of the microneedles being reduced to 0 μm can be understood as complete dissolution. In addition, the molecular weight of the polyglutamic acid used in Table 1 is 500 kDa, and the molecular weight of the pullulan is 300 kDa, but the present invention is not limited to this. In another embodiment, the molecular weight of the polyglutamic acid can be 600 kDa or 700 kDa, and the molecular weight of the pullulan can be 400 kDa.

TABLE 1

| | | | | | | Needle | Dissolving | |
|---|---|---|---|---|---|---|---|---|
| | Ingredient | Ingredient | | | Integrity | structure | effect after | |
| | 1 | 2 | | | after | of the | 2 hours of | Overall |
| NO. | (wt %) | (wt %) | Ratio | Base | demolding | microneedles | application | evaluation |
| 1 | 10 | 0 | — | smooth | abnormal | abnormal | complete | poor |
| 2 | 5 | 0 | — | smooth | abnormal | abnormal | complete | poor |
| 3 | 1.5 | 0 | — | smooth | abnormal | abnormal | complete | poor |
| 4 | 0.75 | 0.75 | 1 | smooth | complete | abnormal | complete | poor |
| 5 | 0.70 | 0.70 | 1 | smooth | complete | abnormal | complete | poor |
| 6 | 0.60 | 0.60 | 1 | smooth | complete | abnormal | complete | poor |
| 7 | 0.50 | 0.50 | 1 | smooth | complete | abnormal | complete | poor |
| 8 | 0.50 | 0.60 | 0.83 | smooth | complete | complete | complete | excellent |
| 9 | 0.50 | 0.75 | 0.67 | smooth | complete | complete | complete | excellent |
| 10 | 0.50 | 1.0 | 0.50 | smooth | complete | complete | complete | excellent |
| 11 | 0.25 | 1.25 | 0.20 | smooth | complete | complete | complete | excellent |
| 12 | 0.20 | 1.30 | 0.15 | smooth | complete | complete | complete | excellent |
| 13 | 0.1 | 1.40 | 0.07 | abnormal | complete | complete | not | poor |
| | | | | | | | complete | |
| 14 | 0 | 1.25 | 0 | abnormal | complete | complete | not | poor |
| | | | | | | | complete | |

From Table 1, it can be seen that when the ratio of the weight of the polyglutamic acid to the weight of the pullulan ranges from 0.1 to 0.9 (the formulas NO. 8-12), the overall evaluation of the produced microneedle device can be regarded as excellent, in which the film surface of the base is smooth, the structure is complete after demolding (e.g., no rupture during demolding or the microneedle device will melt due to moisture after demolding), the needle structure of the microneedles is complete, and the microneedle device can be completely dissolved after 2 hours when application. Therefore, because the ratio of the weight of the polyglutamic acid to the weight of the pullulan ranged from 0.1 to 0.9, the microneedle device 100 of the embodiment of the present invention has a high dissolution rate and manufacturing feasibility.

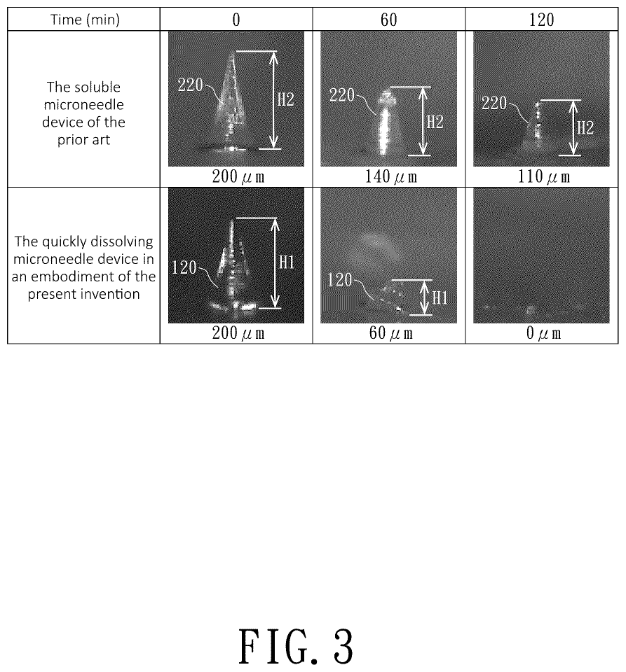

FIG. 3 is the compared result of an experiment with the microneedle device 100 in the embodiment of the present invention versus a soluble microneedle device of the prior art, wherein the microneedle device 100 of the present embodiment (as shown in FIG. 1) is made in the ratio of the polyglutamic acid to the pullulan in No. 9 in Table 1. The experiment involves attaching the microneedle device to the skin, then removing the microneedle device from the skin after a predetermined period of time, and observing the height H of the remaining microneedles, wherein the predetermined period of time is 60 minutes and 120 minutes. It should be noted that the “0 minutes” shown in FIG. 3 is the state when the microneedle device has not yet been attached to the skin. The microneedles 120 of the microneedle device 100 of the present embodiment and the microneedles 220 of the microneedle device of the prior art are, for example, in the conical shape, and the height H1 of the microneedles 120 and the height H2 of the microneedles 220 are both 200 μm before attaching to the skin. It can be seen from FIG. 3 that the height H1 of the microneedles 120 in this embodiment becomes 60 μm after 60 minutes, that is, 70% of the microneedles 120 has been dissolved, in contrast, the height H2 of the microneedles 220 of the prior art becomes 140 μm after 60 minutes. That is, only 30% of the microneedles 220 is dissolved in the prior art. The microneedles 120 of the present embodiment have basically been completely dissolved after 120 minutes, while only 45% of the microneedles 220 of the prior art are dissolved, and there are still 55% of the microneedles 220 have not yet been dissolved. Based on the above, it can be found that the dissolution rate of the microneedle device 100 in the present embodiment is substantially higher than that of the microneedle device of the prior art, which proves that the microneedle device 100 of the embodiment of the present invention has a fast dissolution effect.

In an embodiment of the present invention, the material of the base and the microneedles further includes, for example, a functional ingredient and the functional ingredient is selected from a group of functional ingredients consisting of at least one of hyaluronic acid, salicylic acid, vitamin B5, vitamin C, vitamin E, nicotinic acid, sulfur, Centella asiatica, ceramide, glycerin, GLP-1 receptor agonist, insulin, and acetaminophen. In an embodiment of the present invention, the functional ingredient can be added to the microneedle device 100 according to the requirements. The formula of the microneedle device 100 in the embodiment of the present invention is shown in Table 2. Table 2 presents the weight percentages of the polyglutamic acid, the pullulan, and the optional functional ingredient in the microneedle device 100. The “ratio” is the value obtained by dividing the weight of the polyglutamic acid by the weight of the pullulan sugar, rounded to two decimal places.

TABLE 2

| | | | Functional | |
|---|---|---|---|---|
| | Polyglutamic | Pullulan | ingredient | |
| Embodiment | acid (wt %) | (wt %) | (wt %) | Ratio |
| 1 | 1 | 1.11 | 97.89 | 0.90 |
| 2 | 1 | 10 | 89 | 0.10 |
| 3 | 9 | 90 | 1 | 0.10 |
| 4 | 9.09 | 90.91 | 0 | 0.10 |
| 5 | 10 | 90 | 0 | 0.11 |
| 6 | 45 | 50 | 5 | 0.90 |
| 7 | 47.37 | 52.63 | 0 | 0.90 |

According to Table 2, in the embodiment(s) of the present invention, for example, the weight of the polyglutamic acid accounts for 1% to 50% of the weight of the microneedle device 100, and the weight of the pullulan accounts for 1% to 90% of the weight of the microneedle device 100. In addition, in the embodiments in which the microneedle device 100 includes the functional ingredient (embodiments 1-3 and 6 in Table 2), the weight of the functional ingredient accounts for 1% to 98% of the weight of the microneedle device 100. However, the present invention does not make specific restrictions on this.

In summary, because the ratio of the weight of the polyglutamic acid to the weight of the pullulan ranges from 0.1 to 0.9, the microneedle device of the embodiments of the present invention has a higher dissolution rate and manufacturing feasibility.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle device, comprising:
a base; and
a plurality of microneedles, arranged on a surface of the base,
wherein a material of the base and the microneedles comprises polyglutamic acid and pullulan, and a ratio of a weight of the polyglutamic acid to a weight of the pullulan is ranged from 0.1 to 0.9.

2. The microneedle device according to claim 1, wherein the material of the base and the microneedles further comprises a functional ingredient, and the functional ingredient is selected from a group consisting of at least one of hyaluronic acid, salicylic acid, vitamin B5, vitamin C, vitamin E, nicotinic acid, sulfur, Centella asiatica, ceramide, glycerin, GLP-1 receptor agonist, insulin, and acetaminophen.

3. The microneedle device according to claim 1, wherein the weight of the polyglutamic acid accounts for 1% to 50% of a weight of the microneedle device, and the weight of the pullulan accounts for 1% to 90% of the weight of the microneedle device.

4. The microneedle device according to claim 1, wherein a molecular weight of the polyglutamic acid is between 400 kDa and 1,000 kDa.

5. The microneedle device according to claim 1, wherein a molecular weight of the pullulan is between 200 kDa and 1,000 kDa.

6. The microneedle device according to claim 1, wherein each of the microneedles has a height in a direction perpendicular to the surface, and the height is between 100 μm and 1,500 μm.

7. The microneedle device according to claim 1, wherein each of the microneedles has a height in a direction perpendicular to the surface, and the height is reduced to less than or equal to a half of the height before attachment after the microneedle device is attached to a skin for 60 minutes.

8. The microneedle device according to claim 1, wherein each of the microneedles has a height in a direction perpendicular to the surface, and the height is reduced to less than or equal to one-third of the height before attachment after the microneedle device is attached to a skin for 120 minutes.
